(19) United States
(12) Patent Application Publication
(10) Pub. No.: US 2010/0094198 A1
(43) Pub. Date: Apr. 15, 2010

(54) SYSTEMS AND METHODS FOR TIGHTENING OPHTHALMIC SURGICAL NEEDLES

(76) Inventor: Seth D. Burgett, Glen Carbon, IL (US)

Correspondence Address: Bausch & Lomb Incorporated, One Bausch & Lomb Place, Rochester, NY, 14604-2701, US

(21) Appl. No.: 12/250,602

(22) Filed: Oct. 14, 2008

(51) Int. Cl. A61F 9/007

(52) U.S. Cl. 604/22

(57) ABSTRACT

An ophthalmic surgery system includes a pump for generating fluid flow during ophthalmic surgery, a handpiece interface for providing control of a surgical handpiece, and a power wrench assembly for coupling to an ophthalmic surgical needle. The power wrench assembly is configured to rotate the ophthalmic surgical needle relative to the surgical handpiece until torque applied to the ophthalmic surgical needle is within a desired torque range.

### BACKGROUND

1. Field

The present disclosure is directed to systems and methods for tightening ophthalmic surgical needles onto surgical handpieces.

2. Description of the Related Art

The statements in this section merely provide background information related to the present disclosure and may not constitute prior art.

In eye surgery, particularly phacoemulsification surgery, the use of ophthalmic surgical needles is commonly required in cataract surgeries. Ophthalmic surgical needles transfer ultrasonic energy through a surgical handpiece to a cataract within a patient's eye. It is desired to have proper contact between the ophthalmic surgical needle and the surgical handpiece to provide efficient transmission of ultrasonic energy and reliable connection of the ophthalmic surgical needle. Proper connection between the ophthalmic surgical needle and the surgical handpiece is generally achieved by threading the ophthalmic surgical needle onto the surgical handpiece.

Presently, a user threads an ophthalmic surgical needle onto the surgical handpiece by hand, and then uses a disposable wrench, provided with each ophthalmic surgical needle, to tighten the ophthalmic surgical needle. The disposable wrench is a one-use tool and requires the user to use two hands while tightening the ophthalmic surgical needle onto the surgical handpiece.

### BRIEF DESCRIPTION OF THE DRAWINGS

The drawings described herein are for illustration purposes only and are not intended to limit the scope of the present disclosure in any way.

FIG. 1 is perspective view of an ophthalmic surgery system including a power wrench assembly according to the present disclosure; and

FIG. 2 is a partial elevation view of a handpiece and ophthalmic surgical needle for use with ophthalmic surgery systems according to the present disclosure.

### DETAILED DESCRIPTION OF THE PREFERRED EMBODIMENT

The following description is merely exemplary in nature and is not intended to limit the present disclosure, application, or uses.

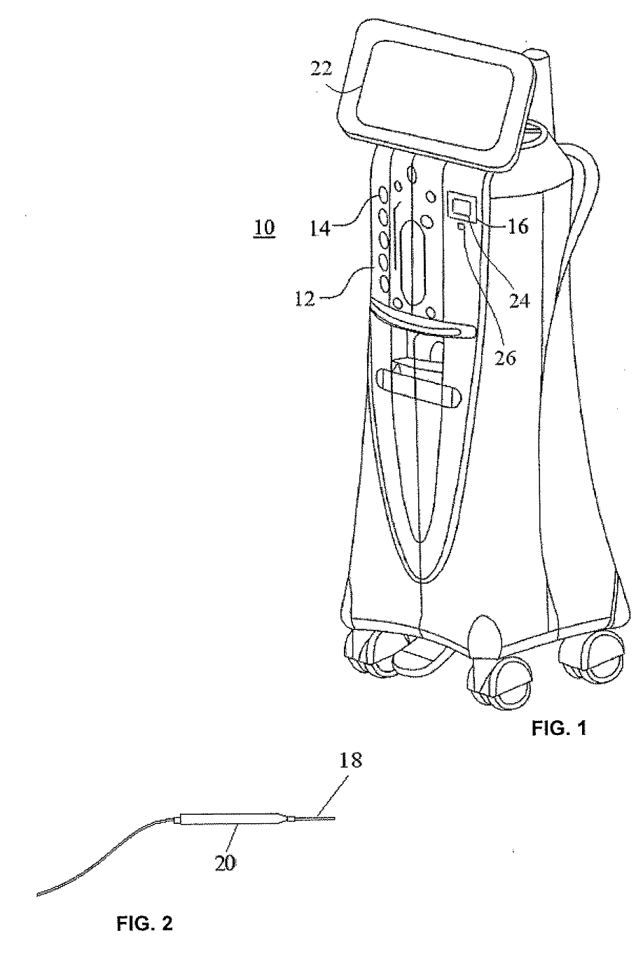

According to one embodiment of the present disclosure, an ophthalmic surgery system 10 is illustrated in FIG. 1. The ophthalmic surgery system 10 includes a pump 12 for generating fluid flow during ophthalmic surgery and a handpiece interface 14 for providing control of a surgical handpiece. The ophthalmic surgery system 10 also includes a power wrench assembly 16 for interfacing an ophthalmic surgical needle at least partially threaded onto the surgical handpiece. One exemplary embodiment of an ophthalmic surgical needle 18 and a surgical handpiece 20 are illustrated in FIG. 2. The ophthalmic surgical needles and surgical handpieces as described herein are generally used for phacoemulsification surgical procedures.

The power wrench assembly 16 is configured to rotate the ophthalmic surgical needle relative to the surgical handpiece until torque applied to the ophthalmic surgical needle is within a desired torque range. In this manner, tightening the ophthalmic surgical needle is automated (i.e., not by a user) thereby reducing potential human errors in achieving the desired torque range and enhancing connection of the ophthalmic surgical needle thereby improving the tuning process of ophthalmic surgery system 10.

Automatically tightening the ophthalmic surgical needle further reduces the overall time needed to prepare the surgical handpiece for surgery. Additionally, the power wrench assembly 16 also permits a user, such as a technician, clinician, nurse, etc., to conveniently tighten an ophthalmic surgical needle onto a surgical handpiece with only one hand. Also, additional tools for use with an ophthalmic surgical needle, e.g., the disposable tools, may be eliminated thereby reducing the cost of each ophthalmic surgical procedure.

In use, a user partially threads an ophthalmic surgical needle onto a surgical handpiece. The surgical handpiece is then positioned proximal to the power wrench assembly 16 with the ophthalmic surgical needle extending into an opening defined by the power wrench assembly 16. In this manner, ophthalmic surgery system 10 eliminates risk of potential puncture injuries to the user during tightening of the ophthalmic surgical needle. With the surgical handpiece positioned proximal to the power wrench assembly 16, the user applies pressure to the surgical handpiece thereby biasing the ophthalmic surgical needle and/or surgical handpiece against the power wrench assembly 16. When the power wrench assembly 16 detects the biasing, the power wrench assembly 16 rotates the ophthalmic surgical needle relative to the surgical handpiece. The power wrench assembly 16 continues to rotate the ophthalmic surgical needle, increasing torque applied to the ophthalmic surgical needle as necessary. When the torque applied to the ophthalmic surgical needle is within a desired range, the power wrench assembly 16 discontinues rotating the ophthalmic surgical needle automatically.

The desired range may be provided by a manufacturer of one or more of the ophthalmic surgical needle, the surgical handpiece, and ophthalmic surgical system. The desired torque range is generally any range sufficient to provide a proper connection between an ophthalmic surgical needle and a handle piece. Further, the desired torque range may also be provided by a user via a user-interface to the ophthalmic surgery system 10 or preprogrammed into the ophthalmic surgery system 10.

The ophthalmic surgery system 10 includes an indicator 22 for providing a visual and audible indication to a user indicating when the torque applied to the ophthalmic surgical needle is within the desired range. While the indicator 22 included in the ophthalmic surgery system 10 is visual and audible, it should be appreciated that various types of audio and/or visual indicators can be employed in other embodiments of the present disclosure. In this embodiment, the indicator 22 is a display associated within the surgery system for providing other information related to the power wrench assembly 16 and/or relevant to the ophthalmic surgery, such as flow information related to the pump 12 or monitoring of the patient. In still other embodiments, an indicator may be omitted, whereby a user may be prompted to remove the surgical handpiece by the power wrench assembly discontinuing rotation of the ophthalmic surgical needle.

When torque applied to the ophthalmic surgical needle is within a desired torque range as provided by the indicator 22, the user may remove the surgical handpiece with the secured ophthalmic surgical needle from the power wrench assembly 16 for use in ophthalmic surgery.

After the ophthalmic surgery, the ophthalmic surgical needle and surgical handpiece are removed from the ophthalmic surgery system 10. For a subsequent ophthalmic surgery, another surgical handpiece is coupled to the ophthalmic surgery system 10 via the handpiece interface 14. An ophthalmic surgical needle is threaded onto the surgical handpiece by the user and presented to the power wrench assembly for tightening. Before the surgical handpiece is positioned proximal to the power wrench assembly 16, a removable wrench insert 24 included in the power wrench assembly 16 is replaced. The removable wrench insert 24 is configured to contact the ophthalmic surgical needle. Contact between power wrench assembly 16 and the ophthalmic surgical needle and surgical handpiece in generally limited to the removable wrench insert 24. By replacing the removable wrench insert 24, a sterile contact is preserved between the power wrench assembly and the ophthalmic surgical needle and the surgical handpiece for the subsequent ophthalmic surgery.

In some embodiments, a removable wrench insert 24 may be reusable when sterilized, e.g., autoclaved, or the insert 24 may be disposable.

Referring again to FIG. 1, the ophthalmic surgery system 10 also includes a sensor 26 for monitoring when a removable wrench insert is removed. Based on an output from the sensor 26, the ophthalmic surgery system 10 may be configured to indicate when replacement of the removable wrench insert is necessary. In the embodiment illustrated in FIG. 1, replacement of the removable wrench insert is generally necessary for each patient. By indicating replacement of the removable wrench insert is necessary, the ophthalmic surgery system 10 is a convenient and reliable indicator to the user to further aid in ensuring a sterilized contact with the power wrench assembly for each patient. Specifically, the inventors hereof have recognized that in some embodiments, an ophthalmic surgery system with a power wrench assembly and a sensor monitoring a removable wrench insert may reduce the potential of reusing unsterilized instruments, e.g., disposable tools, for different patients.

In other embodiments, the ophthalmic surgery system 10 may be configured to indicate replacement of a removable wrench insert is necessary after each use. The particular timing of the indication may vary for different embodiments of the present disclosure. For example, an indication may be provided to a user when the user attempts to tighten a second ophthalmic surgical needle via the power wrench assembly 16 without replacing the removable wrench insert 24. The indication may be displayed via indicator 22 or a different visual and/or audible indicator included in the ophthalmic surgery system 10. For example, an LED may be included adjacent to the power wrench assembly such that the LED is within a users field of view when using the power wrench assembly.

Although several aspects of the present disclosure have been described above with reference to phacoemulsification instruments, it should be understood that various aspects of the present disclosure are not limited to phacoemulsification instruments, and can be applied to a variety of other ophthalmic surgical procedures.

By implementing any or all of the teachings described above, a number of benefits and advantages can be attained including improved reliability, reduced down time, elimination or reduction of redundant components or systems, avoiding unnecessary or premature replacement of components or systems, and a reduction in overall system and operating costs.

We claim:

1. An ophthalmic surgery system comprising:
a pump for generating fluid flow during ophthalmic surgery;
a handpiece interface for providing control of a surgical handpiece;
a power wrench assembly for coupling to an ophthalmic surgical needle; and
wherein the power wrench assembly is configured to rotate the ophthalmic surgical needle relative to the surgical handpiece until torque applied to the ophthalmic surgical needle is within a desired torque range.

2. The ophthalmic surgery system of claim 1, wherein the power wrench assembly is configured to automatically rotate the ophthalmic surgical needle when a user biases at least one of the ophthalmic surgical needle and the surgical handpiece against the power wrench assembly.

3. The ophthalmic surgery system of claim 1, further comprising an indicator for providing a visual and/or audible indication to a user when the torque applied to the ophthalmic surgical needle is within the desired torque range.

4. The ophthalmic surgery system of claim 1, wherein the power wrench assembly includes a removable wrench insert for contacting an ophthalmic surgical needle.

5. The ophthalmic surgery system of claim 4, wherein the removable wrench insert is autoclavable.

6. The ophthalmic surgery system of claim 1, further comprising a sensor for monitoring a removable wrench insert, wherein based on an output from the sensor, the ophthalmic surgery system is configured to indicate to a user when replacement of the removable wrench insert is necessary.

7. The ophthalmic surgery system of claim 1, further comprising an indicator for indicating to a user when replacement of the removable wrench insert is necessary.

8. A method for tightening an ophthalmic surgical needle onto a surgical handpiece via an ophthalmic surgery system, the ophthalmic surgery system including a power wrench assembly, the method comprising:
detecting at least one of the ophthalmic surgical needle and the surgical handpiece; and
automatically rotating the ophthalmic surgical needle relative to the surgical handpiece until torque applied to the ophthalmic surgical needle is within a desired torque range.

9. The method of claim 8, further comprising notifying a user when the torque applied to the ophthalmic surgical needle is within the desired torque range via a visual and/or audible indicator included in the ophthalmic surgery system.

10. The method of claim 8, further comprising notifying a user when replacement of the removable wrench insert is necessary.
